(19) United States
(12) Plant Patent Application Publication
(10) Pub. No.: US 2003/0101500 P1
(43) Pub. Date: May 29, 2003

(54) AGLAONEMA PLANT NAMED 'DYNAMIC RUBY'

(76) Inventor: Pichai Manichote, Bangkok (TH)

Correspondence Address: CHRISTIE, PARKER & HALE, LLP, 350 WEST COLORADO BOULEVARD, SUITE 500, PASADENA, CA, 91105, US

(21) Appl. No.: 09/995,069

(22) Filed: Nov. 27, 2001

(51) Int. Cl. A01H 5/00

(52) U.S. Cl. PLT/376

(57) ABSTRACT

A new and distinct cultivar of Aglaonema named ‘DYNAMIC RUBY’ characterized by its light red mottles on green ovate leaves, compact and well-branched growth habit.

### BOTANICAL CLASSIFICATION

The botanical classification of the new variety is Aglaonema hybrids ‘DYNAMIC RUBY’.

### BACKGROUND OF THE INVENTION

The present invention is directed to a new and distinct variety of Aglaonema, botanically known as Aglaonema hybrids. The varietal denomination of the new variety is ‘DYNAMIC RUBY.’

The new variety was a seedling among a progeny of Aglaonema (rotundum ‘Giant’×tenuipes)×Aglaonema brevispathum, selected by the inventor.

Asexual propagation of the new variety in Nakhon Pathom, Thailand by vegetative cuttings has demonstrated the uniformity and the stability through successive generations of this new Aglaonema.

### SUMMARY OF THE INVENTION

The following traits have been repeatedly observed in the new variety grown under shade conditions in Nakhon Pathom, Thailand which closely approximate those commonly used in commercial horticultural practice.

The features that in combination are believed to distinguish the new variety from other known varieties of Aglaonema include: compact growth habit with freely branching, dense and upright growth; ovate leaves; adaxial leaf surfaces having dark green color with light red mottles.

### COMPARISON WITH OTHER VARIETY

Plants of the new Aglaonema can be compared to plants of the Aglaonema ‘Siam Majesty’ (U.S. Plant patent application No. 09/706,897, Filed: Nov. 6, 2000). Plants of the new Aglaonema differ from plants of ‘Siam Majesty’ by the following combination of characteristics:

1. Whereas the adaxial leaf surfaces of the new variety have light red mottles instead of plain green as found in ‘Siam Majesty’;

2. The mid-rib and veins of the new variety are green instead of red as found in ‘Siam Majesty’; and

3. The leaf tip of the new variety is attenuate while that of the ‘Siam Majesty’ is acuminate.

### BRIEF DESCRIPTION OF ILLUSTRATIONS

The accompanying photographs show the overall appearance of the new variety as nearly correct as it is possible to make in a color illustration of this type. Throughout this specification, color values are based upon the of The Royal Horticultural Society Colour Chart except where common terms of color definition are employed.

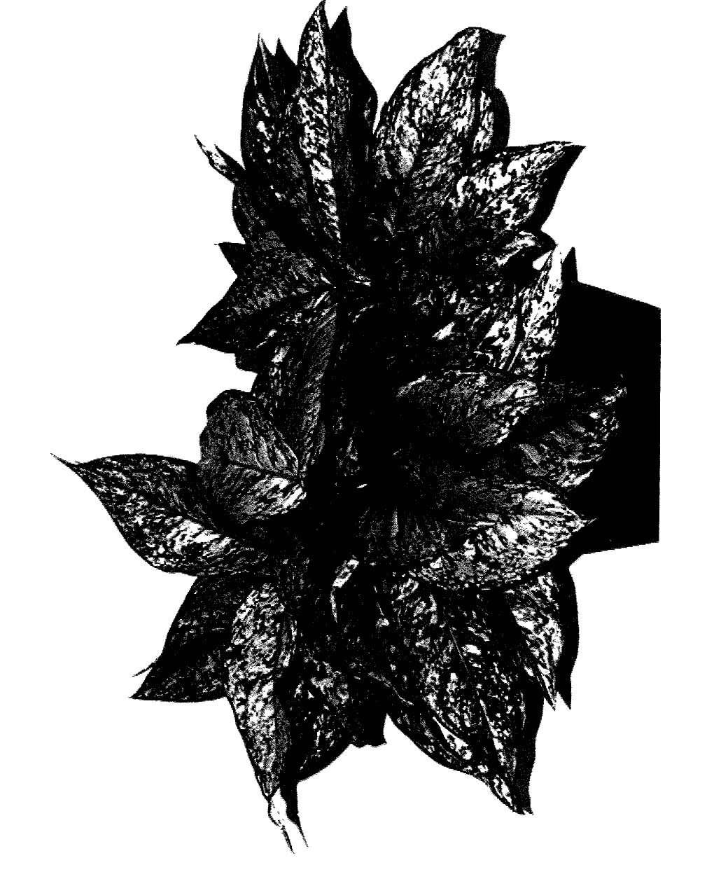

FIG. 1 shows a top view of a single plant of ‘DYNAMIC RUBY’ in a 30 cm container about 15 months after planting;

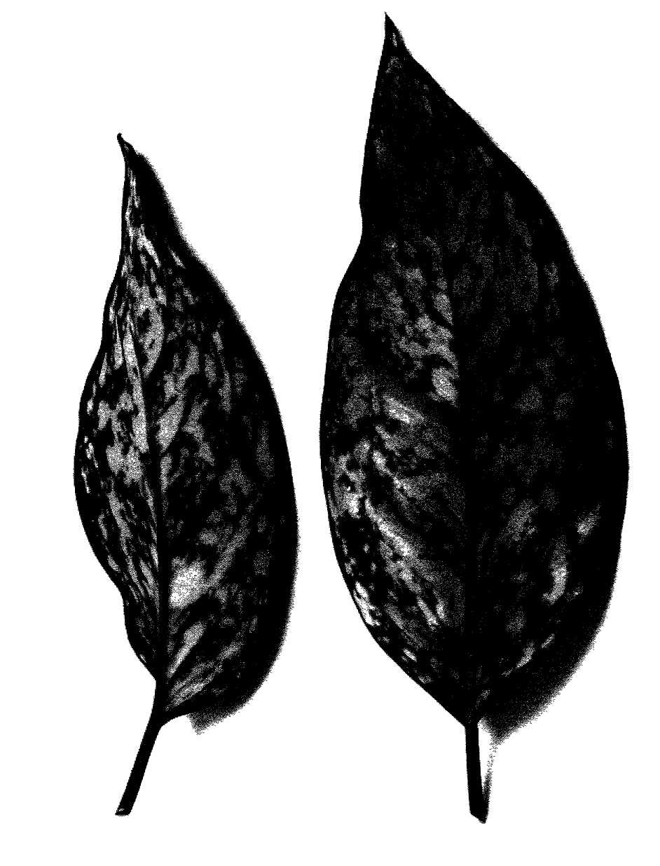

FIG. 2 shows a view of the adaxial surface of a mature leaf of the new variety; and

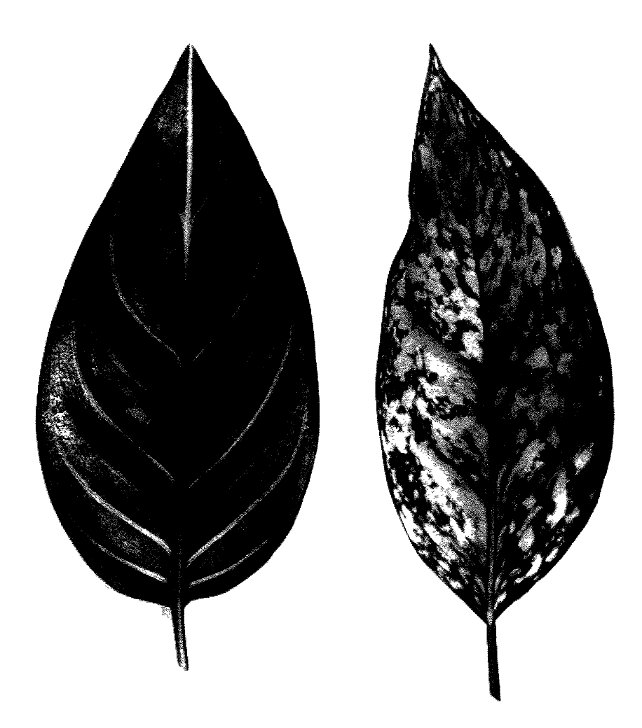

FIG. 3 shows the comparison between the adaxial leaf surface of ‘DYNAMIC RUBY’ and its comparison variety ‘Siam Majesty’.

### DESCRIPTION OF THE NEW VARIETY

All color references are in accordance with The Royal Horticultural Society Colour Chart. Colors are approximate as color depends on climate and environmental condition. Phenotypic expression may vary with environmental, cultural and climatic conditions, as well as differences in conditions of light and soil.

BOTANICAL CLASSIFICATIONS

Aglaonema hybrids.—‘DYNAMIC RUBY’

PARENTAGE

Female parent.—Aglaonema (rotundum×tenuipes)

Male parent.—Aglaonema brevispathum

PROPAGATION: By top cuttings, stem cuttings and by division

PLANT

Shape.—Upright and outwardly spreading, symmetrical, dense and compact

Growth habit.—Erect when young, becoming more outwardly arching and spreading as plant develops

Size.—Height from soil surface to top of leaf canopy about 20 to 29 cm; width about 25 to 33 cm

Vigor.—Vigorous, rapid growth rate

FOLIAGE

Leaf.—

Shape.—Ovate with an attenuate apex, an obtuse base and an entire margin; symmetric leaf blade

Size.—Length about 14.6 to 20.2 cm; width about 7.9 to 9.3 cm

Surface.—Smooth and waxy

Aspect.—Erect initially, eventually horizontal to the stem

Petiole: Length about 2.2 to 3.3 cm Color

Young.—Upper surface near 137D, mottles, near 52A; mid-rib and veins near 145B; lower surface near 137D, mottles near 36C, mid-rib and veins near 145D

Mature.—upper surface near 137C, mottles near 56A, mid-rib and veins near 137A; lower surface near 137C, mottles near 36A, mid-rib veins near 137C

Inflorescence Typical of Aglaonema

Plants of the Aglaonema cultivar ‘Dynamic Ruby’ are dense, compact and have upright growth habit. The upper leaf surfaces are green with light red mottles. The leaves are ovate in shape with attenuate apex. White storage roots are present.

I claim:

1. A new and distinct variety of Aglaonema named ‘DYNAMIC RUBY’ substantially as illustrated and described herein.
